(19) United States
(12) Patent Application Publication
(10) Pub. No.: US 2023/0018807 A1
(43) Pub. Date: Jan. 19, 2023

(54) FLUID MECHANICS-BASED ANALYSIS TOOL TO ESTIMATE FLOW DATA FROM PLANIMETRY CATHETER DATA

(71) Applicant: Northwestern University, Evanston, IL (US)

(72) Inventors: John Erik Pandolfino, Wilmette, IL (US); Neelesh Ashok Patankar, Buffalo Grove, IL (US); Shashank Acharya, Evanston, IL (US); Sourav Halder, Evanston, IL (US); Wenjun Kou, Rockford, IL (US); Peter James Kahrilas, Chicago, IL (US); Dustin Allan Carlson, Chicago, IL (US)

(21) Appl. No.: 17/778,340

(22) PCT Filed: Nov. 19, 2020
(86) PCT No.: PCT/US2020/061388

§ 371 Date: May 19, 2022

Related U.S. Application Data
(60) Provisional application No. 62/937,363, filed on Nov. 19, 2019.

(51) Int. Cl. A61B 5/00; A61M 25/10

(52) U.S. Cl. CPC A61B 5/6853; A61M 25/10; A61B 5/1076

(57) ABSTRACT

A fluid mechanics-based analysis tool is implemented to compute pressure field data, fluid velocity data, and/or muscular work data in a lumen or other tubular organ or structure from planimetry and pressure data (e.g., measured using a balloon dilation or other planimetry catheter). In this way, flow data can be estimated, which are otherwise insensible from current planimetry catheter technologies due to economic and/or manufacturing limitations.

### CROSS-REFERENCE TO RELATED APPLICATIONS

This application claims the benefit of U.S. Provisional Patent Application Ser. No. 62/937,363, filed on Nov. 19, 2019, and entitled “FLUID MECHANICS-BASED ANALYSIS TOOL TO ESTIMATE FLOW DATA FROM PLANIMETRY CATHETER DATA,” which is herein incorporated by reference in its entirety.

### STATEMENT REGARDING FEDERALLY SPONSORED RESEARCH

This invention was made with government support under DK079902 and DK117824 awarded by the National Institutes of Health and under ACI-1450374 awarded by the National Science Foundation. The government has certain rights in the invention.

### BACKGROUND

Balloon dilation catheters are used to inquire about the mechanical state of the tissue in tube-shaped organs and vessels like the esophagus, arteries, cervix, and so on. In these devices, the balloon is usually filled with a fluid like saline. Current dilation catheters are able to measure the cross-sectional area at multiple locations along the tube length, but are limited to measuring the fluid pressure at only one or two locations, usually located at either ends of the tube. During muscular activity, the pressure distribution in the balloon is significantly altered, but is not sensed by the dilation catheter device. The exact pressure distribution, along with the local tube area and fluid velocity, can reveal valuable information about the effort that is expended by the muscle or tissue wall to achieve its biological function. Currently, the fluid velocity is not measurable by any of the sensors housed on conventional dilation catheters.

There is a need to compute detailed pressure and velocity distribution data because they can be direct indicators of the efficiency or strength of muscular contraction in the organ.

### SUMMARY OF THE DISCLOSURE

The present disclosure addresses the aforementioned drawbacks by providing a method for generating flow data from planimetry data acquired with a planimetry catheter. The method includes accessing planimetry data and pressure data with a computer system, where the planimetry data and pressure data were acquired with a planimetry catheter. The planimetry data and the pressure data are input to a reduced order model using the computer system, generating output as flow data. These flow data can be stored for later use or display to a user. Muscular work data can also be generated from the flow data, which may include pressure field data indicating pressure measurements along the extent of the planimetry catheter and fluid velocity data.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 shows an example of a planimetry catheter, which in this example includes a balloon dilation catheter.

FIGS. 2A and 2B show an example visualization of data acquired with a planimetry catheter.

FIG. 3 is a cross-sectional view of an example planimetry catheter device inside the esophagus. The left panel shows the device within the infinite compliance limit. The right panel shows the bag walls fully taut. The perimeter of the blue curve is equal in both figures.

FIG. 4 shows the shape of a balloon dilation planimetry catheter bag and contraction zone during peristaltic activity.

FIG. 5 is a flowchart setting forth the steps of an example method for generating pressure field data, fluid velocity or other flow data, and/or muscular work data from planimetry and pressure data using a reduced order model.

FIG. 6 shows an example plot visualizing a peristaltic activation wave.

FIGS. 7A-7D show examples of tube deformations in different regimes.

FIG. 8 shows example geometry in peristaltic contraction as observed in the wave's frame of reference.

FIGS. 9A-9D show examples of regime maps obtained from a reduced order model.

FIGS. 10A-10C show examples of work expenditure curves for different regimes.

FIG. 11 shows an example of pressure and work computations from data acquired with a planimetry catheter.

FIG. 12 is a block diagram of an example pressure field and flow data generating system that can be implemented in accordance with some embodiments described in the present disclosure.

FIG. 13 is a block diagram of components that can implement the system shown in FIG. 12.

### DETAILED DESCRIPTION

Described here are systems and methods for using planimetry and pressure data (e.g., measured using a balloon dilation or other planimetry catheter) to compute pressure field data, fluid velocity data, and muscular work data in a lumen or other tubular organ or structure. In general, these systems and methods provide a fluid mechanics-based analysis tool that can estimate flow data that are otherwise insensible from current balloon dilation catheter technologies due to economic and/or manufacturing limitations.

Advantageously, the systems and methods described in the present disclosure improve existing balloon dilation catheter systems by enabling the measurement and reporting of quantities (e.g., pressure field, fluid velocity, muscular work) that would otherwise require the addition of several sensors to the catheter. As such, the construction of such balloon dilation catheters can be kept simple, and then augmented using the systems and methods described in the present disclosure, thereby keeping manufacturing costs low while significantly increasing the clinical value of the dilation catheter system.

Because the systems and methods described in the present disclosure provide analysis tools that are based on fluid mechanics, they are capable of providing estimates not only for the pressure distribution within the device, but also the fluid velocity as well. These quantities can then be combined in a specific way to reveal the amount of work done by the muscle wall to move fluid, such as swallowed food or blood. This technique can be used to compute new metrics for “work” or “muscular effort” that have the potential to better predict the possibility of disease or dysfunction in the organ. In the esophagus, the systems and methods add the ability to sense via computation the values of fluid pressure, velocity, and work during peristalsis. In blood vessels, the systems and methods are able to sense via computation the pressure drop due to blood flow across narrowing geometries like clots or swollen tubular walls due to aneurysm.

In this way, the systems and methods described in the present disclosure can utilize data captured by standard planimetry catheters during clinical use, from which quantities such as fluid pressure, velocity, and overall muscular effort can be estimated at multiple locations in the tube without requiring any design modifications or addition of further sensors to the catheter. As such, the systems and methods described in the present disclosure can be implemented with dilation catheter or other suitable catheter devices without requiring a redesign of those devices.

As another advantage, the systems and methods described in the present disclosure implement fast computations that enable the analysis tools to be used at the point-of-care without significant delays.

Additionally or alternatively, the systems and methods can provide additional visualization tools for clinicians to understand the flow patterns inside the balloon due to muscular activity.

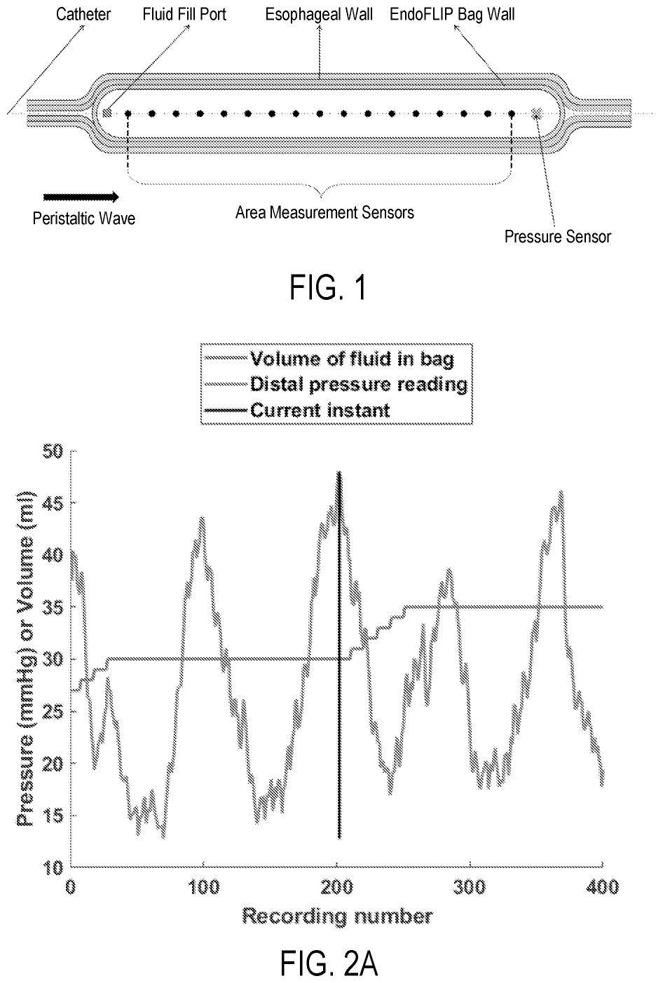

An example of a balloon dilation catheter is shown in FIG. 1. For instance, the balloon dilation catheter can be an endolumenal functional lumen imaging probe (EndoFLIP; Crospon, Ireland) or other such device capable of measuring planimetry and pressure data. The dilation catheter includes a long, flexible, hollow wire at the end of which is mounted a compliant balloon, which may be composed of a polyurethane bag, for instance. When deployed during clinical procedures, the device is positioned in such a way that the balloon rests within the esophageal lumen. The balloon is inflated using saline and the response of the lumen wall is evaluated by monitoring the internal fluid pressure. Various versions of the device exist where the balloon length can be, for example, 8 cm, 16 cm, etc.

When fully inflated with saline, the diameter of the balloon can be on the order of tens of millimeters, such as 22 mm. The section of the catheter enclosed by the balloon includes several planimetry sensors that can measure the area along the entire balloon length. The bottom tip of the catheter can house a pressure sensor that measures the fluid pressure within it at any given time. The area measured by the sensors is based on the assumption that the cross-section of the tube at every point along the axis is circular. The other end of the catheter is connected to a computer that stores the data collected by these sensors. The balloon can be filled or drained by pumping fluid through the hollow channel within the catheter. Fluid enters or leaves the balloon through a small hole on the catheter wall.

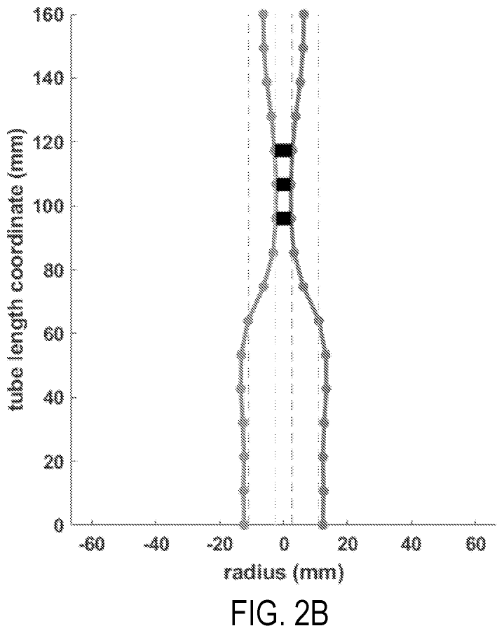

As an example, the data collected with a dilation catheter system can be visualized as shown in FIGS. 2A and 2B. The graphs in FIG. 2A show the pressure and volume inside the tube as a function of time. The volume change is controlled by the physician conducting the procedure and that the pressure change is a consequence of peristaltic activity and the wall's response to distension. FIG. 2B shows the axisymmetric profile of the tube at a certain instant of time, represented by the black vertical line in FIG. 2A. The dotted lines superimposed on the tube profile show the maximum and minimum area that can be accurately measured by the planimetry sensors. The contraction (highlighted with the three black bands) is detected by finding the location along the tube length where the area is the smallest.

From the pressure curve, four peaks can be seen. These peaks indicate that four peristaltic waves have passed over the tube length, one after the other, for the duration of this plot. The plot of the tube profile is oriented in such a way that peristalsis begins at the top and travels downwards. The measurement of area is not continuous along the tube length. For example, seventeen sensors span the tube length and the area is polled at 1 cm intervals along the tube length in between each pair of sensors. The dots along the tube profile curves represent the location where the area was captured and interpolation between these locations completes the construction of the tube profile for visualization.

The device is primarily located in the esophageal lumen during operation; however, other balloon dilation catheters could be configured for use in other tubular organs or bodily lumens. The passage of a peristaltic wave causes the (axisymmetric) profile of the balloon to change and is accompanied by a change in fluid pressure within.

The balloon walls have a fixed perimeter at each point along the tube length. As such, when partially filled, the walls remain unstretched, folded and are said to be within the “infinite compliance limit.” In this state, the bag walls do not resist the introduction of additional fluid and any resultant pressure rise within the bag is solely due to stretching of the esophageal walls. After a sufficient amount of fluid is introduced into the balloon, the walls finish unfolding and begin to get taut. At this stage, the pressure inside begins to sharply rise. The balloon is not in the infinite compliance limit anymore and strongly resists further introduction of fluid.

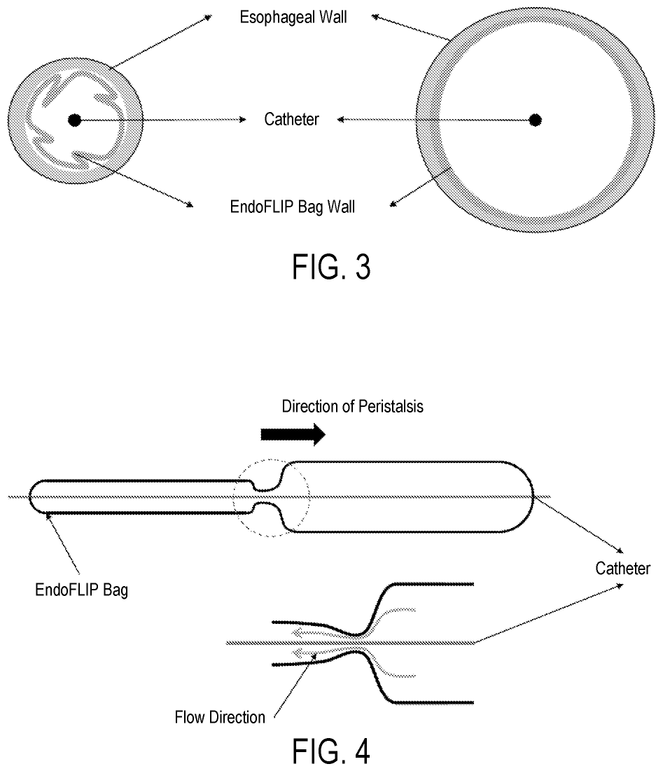

Within the infinite compliance limit, the pressure in the balloon is a function of the esophageal (or other lumen) wall's mechanical properties but outside of it, the pressure also depends on the stiffness of the balloon wall material. FIG. 3 further illustrates the shape of the bag wall within and outside the infinite compliance limit.

During peristalsis, the balloon walls approach the surface of the catheter and occasional contact is possible. FIG. 4 shows the geometry and possible flow directions within the contraction zone. The flow through this narrow zone occurs in the space between the catheter and the balloon surface. Combined with the increased and irregular surface area due to the folds, it can be difficult to predict the relationship between flowrate and pressure drop across the contraction zone.

If the flow inside is assumed to be similar to pipe flow at every location, then the flow is laminar. But the Reynolds number inside the contraction zone can be difficult to estimate so the nature of the flow at the neck is unknown. As such, two flow types can be analyzed: one in which a simple friction factor can be used to relate flowrate and pressure drop in the entire domain, and one in which flow is parabolic (e.g., laminar) everywhere. In both cases, the presence of the catheter can be ignored such that the flow cross section can be estimated as circular instead of annular. The framework of the model described in the present disclosure can be adapted to account for these complications without significantly altering the observations.

As described in the present disclosure, a model to analyze this system and understand the relationship between the tube profile, internal pressure, esophageal (or other lumen) wall stiffness, and intensity of peristaltic contraction. The effort (i.e., “work”) put in by the walls to pump fluid within the balloon can also be computed or otherwise estimated. Using this information, the health of peristaltic activity in the esophagus (or other lumen) and the mechanical state of the muscle wall during swallowing, which is invaluable for diagnosing dysphagia, can be assessed.

In general, planimetry and pressure data acquired with a balloon dilation catheter or other suitable measurement device are enhanced and processed to systematically compute the pressure and velocity readings at all locations along the tube length. Based on the principle of energy conservation, a metric for “muscular effort” or “active work” can also be computed.

The planimetry and pressure data can be enhanced by increasing the resolution of these data. Additionally or alternatively, the noise from planimetry sensors can be eliminated or otherwise reduced such that the planimetry data can be treated as a time varying input along with the pressure reading. The systems and methods described here can then produce a visualization of the spatiotemporal flow velocity and pressure field fields within the catheter balloon. The newly computed pressure and velocity distributions can be combined along with the planimetry data to estimate the value of active, passive, and viscous work done during a muscular contraction event. These values can then be examined to provide an assessment on the physiological state of muscular or tissue contraction in the tubular organ.

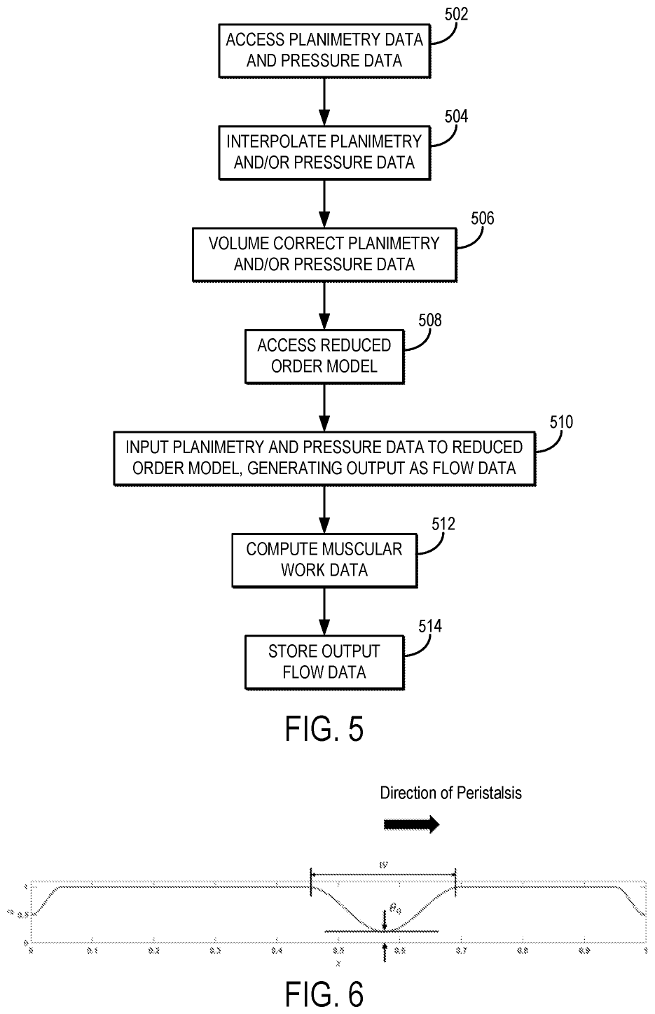

Referring now to FIG. 5, a flowchart is illustrated as setting forth the steps of an example method for generating pressure field data, fluid velocity data, and muscular work data from planimetry and pressure data measured with a balloon dilation catheter or other planimetry catheter.

The method includes accessing planimetry data and pressure data with a computer system, as indicated at step 502. Accessing the planimetry and pressure data with the computer system can include retrieving such data from a memory or other suitable data storage device or medium. Additionally or alternatively, such data can be accessed with the computer system by acquiring the data with a suitable measurement device (e.g., a balloon dilation catheter) for measuring planimetry and pressure data in a lumen. These acquired data can then be communicated to the computer system, which may be done in real-time as data are acquired or after the data have been acquired.

As an example, planimetry data can include measurements of distances, angles, areas, and other geometric quantities. For instance, the planimetry data can include measurements of internal diameter, cross-section, and/or distensibility of a lumen, vessel, or other hollow organ. As an example, the pressure data can include pressure measured at a point location, such as at the proximal end of a balloon dilation catheter. In other instances, the pressure data can include a measurement of pressure at another location, such as the distal end of the balloon dilation catheter, or at another point along the length of the balloon dilation catheter. In some instances, the pressure data may also include measurements of pressure at more than one location.

The planimetry data, pressure data, or both, may be interpolated to increase the sampling density of the measured parameters, as indicated at step 504. In some implementations, a volume correction can be applied to the planimetry data, pressure data, or both, as indicated at step 506. As a non-limiting example, the volume correction can use a bag volume (“By”) reading measured by the pump system as a basis for the volume correction.

A reduced order model is accessed with the computer system, as indicated at step 508. The reduced order model can be accessed by retrieving a previously constructed model from a memory or other data storage device or medium. In other instances, the reduced order model can be accessed with the computer system by generating or otherwise constructing the model.

In general, the reduced order model includes one or more equations based on fluid dynamics and is able to predict fluid flow and the resultant tube wall deformation in instances such as when a peristaltic wave passes over a closed cylindrical tube. An example of a reduced order model that can be implemented is described below in more detail. In some implementations, the effect of nonlinear tube laws on the value of work done during peristalsis can be incorporated into the model. For instance, material properties along the length of the tube may be allowed to vary. As another example, the modeled peristaltic activation wave can be allowed to have a non-constant velocity, such that the peristaltic wave can speed up or even completely stop depending on the amount of obstruction it senses.

The planimetry data and the pressure data are then input to the reduced order model, as indicated at step 510. Inputting the planimetry and pressure data to the reduced order model generates output as pressure field data, fluid velocity data, other flow data, or combinations thereof. Pressure field data includes measurements of pressure along the full extent of the balloon dilation catheter, rather than only at the location(s) of the pressure sensor (s). Fluid velocity data includes measurements of fluid velocity through the lumen. In this way, the analysis provided effectively enables the balloon dilation catheter device to behave as though it has multiple pressure and velocity sensors housed on the catheter.

Using the pressure field and fluid velocity, or other flow, data, muscular work data are computed, as indicated at step 512. These muscular work data indicate a quantification of work done by muscle associated with the tubular organ, such as peristaltic work done by the esophagus during a swallow action. In this way, the muscular work data can be analyzed to assess work done by the subject during a swallow and to thereby assess the subject's capability to propel fluid. This quantitative information can provide valuable insights into swallow function for a clinician.

In addition to generating muscular work data, one or more regime maps can be generated a posteriori after several data points have been collected from tests over a certain window of time or from several subjects, as described below in more detail.

The computed data can be stored as output, as indicated at step 514. Storing the output can include storing the pressure field data, fluid velocity data, other flow data, and/or muscular work data on a memory or other suitable data storage device or medium. In other instances, storing the output can include displaying one or more of the pressure field data, fluid velocity data, other flow data, and/or muscular work data to a user. For example, maps, images, or plots of the data can be generated and displayed to a user.

Having described an example method for generating pressure field data, fluid velocity or other flow data, and/or muscular work data based on inputting planimetry and pressure data to a reduced order model, examples of such models are now described.

Flow in a tube with deforming walls can be modeled using the system of equations given by,

$$\frac{\partial A}{\partial t} + \frac{\partial ( Au )}{\partial x} = 0 ; \qquad ( 1 )$$

$$\frac{\partial u}{\partial t} + u \frac{\partial u}{\partial x} = - \frac{1}{ρ} \frac{\partial p}{\partial x} - \frac{2 {τ}_{R}}{ρ R} ; \qquad ( 2 )$$

where A(x,t) is the tube area and u(x,t) is the fluid velocity. The axial coordinate along the tube length is denoted by x.

To close the system of equations, it can be assumed that a direct relationship between pressure, p, and area, A, exists. This relationship is commonly referred to as a “tube law.” As a non-limiting example, it can be assumed that the change in pressure is proportional to the change in area. The form of this tube law, in which it is assumed that the external pressure is zero for all time and thus the transmural pressure is equal to the pressure inside the tube, can be given by,

$$p = K ( \frac{A}{{A}_{0}} - 1 ) + Y \frac{\partial A}{\partial t} . \qquad ( 3 )$$

where K is a measure of the wall stiffness and has the units Pascal (Pa). The value for K will depend on the Young's modulus of the muscle wall and the ratio of its thickness to the undeformed radius. A damping term with coefficient Y is also introduced. By a simple manipulation using the continuity equation, the introduction of this term leads to a diffusion term in the momentum equation. Thus, the addition of this term helps stabilize the numerical solution.

$$p = K ( \frac{A}{{A}_{0}} - 1 ) + Y \frac{\partial A}{\partial t} = K ( \frac{A}{{A}_{0}} - 1 ) - Y \frac{\partial ( Au )}{\partial x} ; \qquad ( 4 )$$

$$\frac{\partial p}{\partial x} = \frac{K}{{A}_{0}} \frac{\partial A}{\partial x} - Y \frac{{\partial }^{2} ( Au )}{\partial {x}^{2}} . \qquad ( 5 )$$

It is also noted that there is a viscous shear stress term in the momentum equation. Several approximations can be made to write R as a function of tube area and local fluid velocity. For example, one of the following expressions could be used:

$$\frac{2 τ}{ρ R} = \{ \begin{matrix}f \frac{u \vert u \vert}{2 D} & based on the Darcy friction factor \\ \frac{8 π μ u}{ρ A} & assuming parabolic flow everywhere \\ \frac{2 π μ}{ρ A} ( \frac{Ru}{{δ}_{BLT}} ) & flat profile with a linear boundary layer\end{matrix} ;$$

where δ_{BLT} is the boundary layer thickness; D and R are the diameter and radius of the tube, respectively; and A=πR². Each of these terms can have a quantitative effect on the final solution, but the qualitative effect is similar. As mentioned above, in some instances an expression that uses a friction factor to estimate the viscous flow stress due to the uncertainty introduced by the presence of folds and irregularities in the balloon wall during normal operation can be used, and such a system can be examined if the flow is assumed to be parabolic everywhere.

In a non-limiting example, the following expressions for each of the relevant dimensional variables can be used to obtain the non-dimensional versions of the continuity, momentum, and the tube law equations:

$$A = α {A}_{0} ; \\ t = τ \frac{{L}_{tube}}{c} ; \\ u = Uc ; \\ p = PK ; and \\ x = χ {L}_{tube} .$$

The speed of the peristaltic wave is denoted by c and L_{tube} is the length of the balloon. Substituting these into Eqns. (1)-(3) gives,

$$\frac{\partial α}{\partial τ} + \frac{\partial ( α U )}{\partial χ} = 0 ; \qquad ( 6 )$$

$$\frac{\partial U}{\partial τ} + U \frac{\partial U}{\partial χ} + ψ \frac{\partial P}{\partial χ} + β \frac{U \vert U \vert}{\sqrt{α}} = 0 ; \qquad ( 7 )$$

$$P = ( α - 1 ) - η \frac{\partial ( α U )}{\partial χ} ; \qquad ( 8 )$$

where the following non-dimensional numbers emerge:

$$η = \frac{Y}{K ( \frac{{cA}_{0}}{{L}_{tube}} )} ; \\ ψ = \frac{K}{ρ {c}^{2}} ; and \\ β = ( \frac{{fL}_{tube}}{4} ) \sqrt{\frac{π}{{A}_{0}}} .$$

In some implementations, the non-dimensional pressure, P, in the momentum equation (e.g., Eqn. (7)) can be replaced with the right-hand side of Eqn. (8). In these instances, the total number of coupled equations to be solved is two, with α and U as the unknowns. When working with the laminar, parabolic flow version of the momentum equation, the non-dimensional number for flow resistance, β, becomes 8πμL_{tube}/(ρA₀c) and ψ remains unchanged.

During normal operation, the fluid volume inside the balloon is constant because saline pumping is halted and the balloon ends are sealed. Thus, the fluid velocity at both ends is zero. The balloon is attached to the catheter at both ends and tapers off to a point. In this scenario, the velocity boundary conditions,

U(χ=0,τ)=0 and U(χ=1,τ)=0 (9);

are straightforward, but the boundary conditions for area A may be unclear. Fixing the area to some nonzero constant value is equivalent to fixing the pressure and can therefore lead to an inconsistent problem definition. Thus, the following boundary conditions can be chosen for area, which are equivalent to specifying zero pressure gradients at either ends of the tube:

$$\frac{\partial α}{\partial χ} {\vert}_{χ = 0 , τ} = 0 and \frac{\partial α}{\partial χ} {\vert}_{χ = 1 , τ} = 0. \qquad ( 10 )$$

It should be noted that more complicated forms of the tube law can be used which account for longitudinal curvature, bending, and tension in the tube wall. Such a tube law can include a double derivative, Aₓₓ, or a fourth order derivative, Aₓₓₓₓ, term of the tube area. Under such a setting, additional boundary conditions for area can be applied in a straightforward manner without leading to inconsistencies in the problem specification. The inclusion of these terms will generally lead to higher order (e.g., sixth degree) spatial derivatives into the governing equations.

Another component of the model is specifying an activation input to mimic peristaltic contraction, such as inducing a reduction in the tube area at some location and at some time in a manner that resembles a traveling wave. As one example, contractions can be induced in the model using an external activation pressure at a specific location, which is varied sinusoidally with time. This is an appropriate method of activation for valveless pumping scenarios due to the fact that the extra pressure is generated due to respiration. But in the esophagus, contractions in area are generated due to a contraction of muscle fibers in the esophageal wall. As such, in these applications an activation term that changes the reference area of the tube when activated is added to the tube law. The modified form of the tube law with an activation term is,

$$P = ( \frac{α}{θ} - 1 ) - η \frac{\partial ( α U )}{\partial χ} ; \qquad ( 11 )$$

with the activation term given by,

$$θ = \{ \begin{matrix}1 - ( \frac{1 - {θ}_{0}}{2} ) ( 1 + sin ( \frac{2 π}{w} ( χ - τ ) + \frac{3 π}{2} ) ) , & τ - w \leq χ \leq τ \\ 1 , & otherwise\end{matrix}$$

The non-dimensional width of the peristaltic wave is denoted by w=W/L_{tube}, with W denoting the actual, dimensional width of the contraction. As mentioned earlier, the area at the tube ends in the device tapers off, so a small correction can be made to the activation term to account for the reduced area at the ends. An example plot of such an activation wave is shown in FIG. 6.

The value of θ₀ represents the reference area of the tube at the strongest part of the contraction. The smaller the value of θ₀, the smaller is the reference area and higher is the contraction intensity. When θ₀=1, there is no contraction and the entire system is at rest. When the velocity scale is chosen to be the same as the speed of the peristaltic wave, in the non-dimensional framework, the speed of activation is 1.0.

The non-dimensional pressure term, P, in the momentum equation can be eliminated using the tube law given by Eqn. (11). The final system of equations in matrix form is,

$$[ \begin{matrix}{α}_{τ} \\ {U}_{τ}\end{matrix} ] = [ \begin{matrix}0 \\ η {ψ ( α U )}_{χχ}\end{matrix} ] + [ \begin{matrix}- U \frac{\partial α}{\partial χ} - α \frac{\partial U}{\partial χ} \\ - U \frac{\partial U}{\partial χ} - β ( \frac{U \vert U \vert}{\sqrt{α}} ) - ψ ( \frac{1}{θ} \frac{\partial α}{\partial χ} - \frac{α}{{θ}^{2}} \frac{\partial θ}{\partial χ}\end{matrix} ]$$

with boundary conditions as given in Eqns. (9) and (10) and zero velocity initial conditions and some initial condition for area that indicates the amount of volume inside the tube,

U(χ,τ=0)=0 and α(χ,τ=0)=α_{IC} (12).

This set of equations can be solved using, for instance, traditional two-step schemes. Another approach that is simpler to implement is to add an artificial diffusion term, ε(αₓₓ), to the continuity equation and use a routine such as the “PDEPE” routine in MATLAB to obtain the numerical solution.

When η=0, this system becomes strictly hyperbolic. Adding an artificial diffusion term is can be used to obtain the solution for a hyperbolic system of equations. As a non-limiting example, the value of c can be set as ε=10⁻⁴ and η can be set as η=10⁻³.

For the sake of clarity, the governing equations and the boundary conditions can be rewritten in a form used by the PDEPE routine in MATBAL, which also clearly show the flux terms:

$$[ \begin{matrix}{α}_{τ} \\ {U}_{τ}\end{matrix} ] = \frac{\partial }{\partial χ} [ \begin{matrix}ε {α}_{χ} \\ η {ψ ( α U )}_{χ}\end{matrix} ] + [ \begin{matrix}- U \frac{\partial α}{\partial χ} - α \frac{\partial U}{\partial χ} \\ - U \frac{\partial U}{\partial χ} - β ( \frac{U \vert U \vert}{\sqrt{α}} ) - ψ ( \frac{1}{θ} \frac{\partial α}{\partial χ} - \frac{α}{{θ}^{2}} \frac{\partial θ}{\partial χ} )\end{matrix} ] ; \qquad ( 13 )$$

$$[ \begin{matrix}0 \\ U\end{matrix} ] + [ \begin{matrix}1 \\ 0\end{matrix} ] [ \begin{matrix}ε {α}_{χ} \\ η {ψ ( α U )}_{χ}\end{matrix} ] = [ \begin{matrix}0 \\ 0\end{matrix} ] . \qquad ( 14 )$$

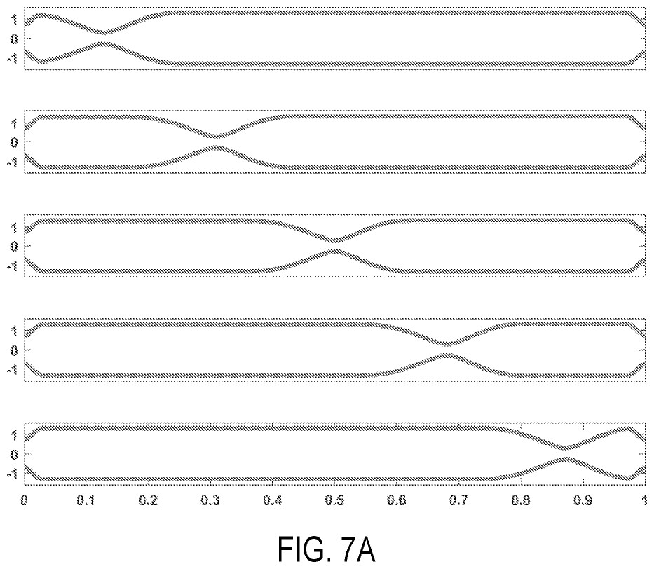

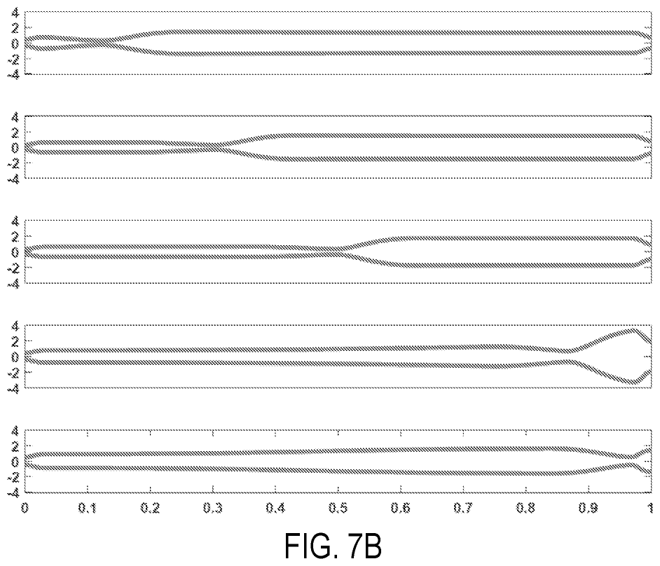

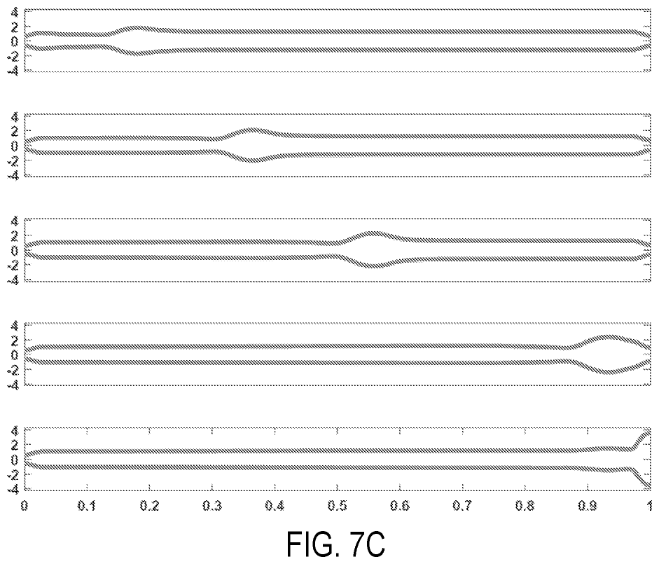

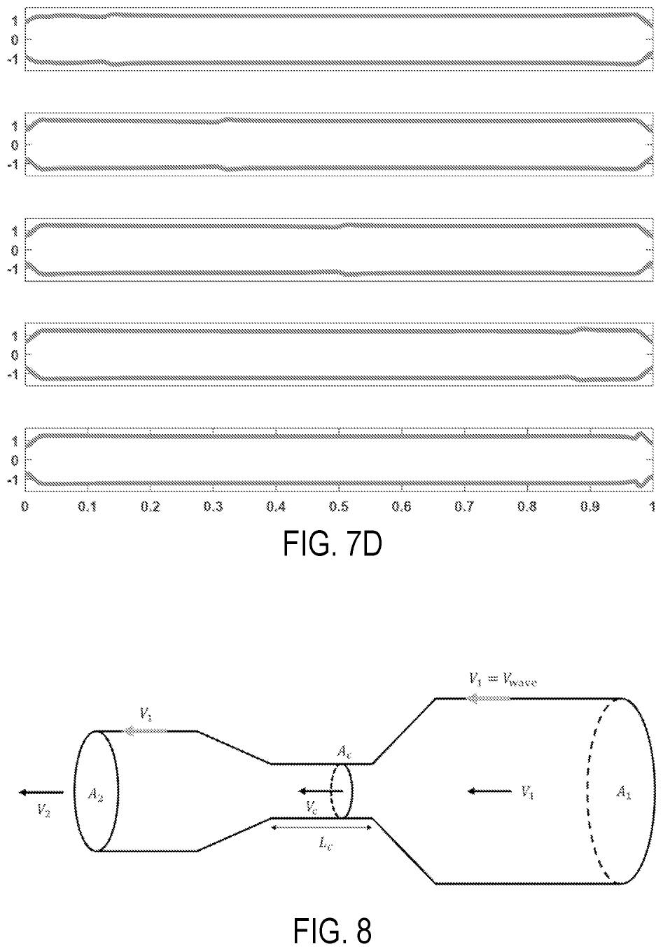

With all the parts of the 1D model in place, the system's response to the applied activation as a function of the operating parameters, θ₀, ψ, and β, can be described. Changing these values is equivalent to investigating the effect of tube wall stiffness, wave speed, contraction strength, fluid density, and flow resistance on the tube wall deformation and internal flow patterns during peristalsis. Based on the system's behavior for a broad range of operating values, four distinct, physiologically relevant patterns of peristaltic pumping can be defined based on the way the tube walls and the fluid inside respond to the applied activation. These regimes are shown in FIGS. 7A-7D. The progression of these regimes from numbers 1 to 4 can be interpreted as the response of the system as fluid viscosity is continually increasing. It should be noted that the fourth regime displays very little deformation of tube area. The deformation dynamics for this regime are generally unremarkable and the tube shape remains quite similar to the shape of the initial condition for the entire duration of wave travel.

The occurrence of these regimes is dictated by a competition between elastic forces generated due to deformation of the tube wall and resistance to flow through the narrowest part of the contraction. A relatively stiff tube wall resists deformation and forces the fluid that was displaced due to the advancing wave to flow back through the contraction. In this scenario, the walls deform as shown in Regime 1 (FIG. 7A). An equivalent way of looking at this is to assume a low resistance to flow through the contraction. The energy required to expand the tube walls is significantly higher than the viscous loss through the contraction and the pumping is almost quasi-steady with the tube walls having the same diameter on either side of the contraction. On the other hand, if the resistance to flow is high, it is favorable for the system to expand the tube walls to accommodate the fluid that is being displaced by the advancing peristaltic wave. At moderate flow resistances, this situation occurs when the stiffness of the tube walls is extremely low. The tube deformation pattern in this scenario is denoted as Regime 2 (FIG. 7B).

In Regime 3 (FIG. 7C), the formation of a “blister” can be seen, as predicted when the resistance to flow is high and the tube walls are fairly compliant. Regime 4 (FIG. 7D) occurs when the resistance to flow is much higher than that in Regime 3. In this case, the amount of time it takes for the fluid to “move” and respond to the peristaltic contraction is much higher than the amount of time it takes for the wave to travel over a section of the tube. The fluid velocity in this case is extremely low leading to a small value in ∂A/∂t as well.

Various patterns of tube wall deformation can be observed depending on the operating conditions of the system. These various operating conditions can be combined in a sensible manner to model their cumulative effect on the system and predict the transition between regimes as a function of these collapsed variables. To that effect, a wave-frame approach can be utilized to analyze peristaltic flow. As a non-limiting example, based on the configuration shown in FIG. 8, the contraction will appear stationary in a reference frame that is attached to the peristaltic wave and the walls of the tube will appear to move with the speed of the peristaltic wave, but in the opposite direction. The fluid velocities in this wave reference frame are represented by black arrows in FIG. 8 and the velocity of the wall is represented with blue arrows in FIG. 8. In some instances, it can be assumed that the velocity of the fluid ahead of the contraction zone, in the lab frame, is zero (this assumption may be incorrect if the flow is laminar and parabolic but is valid when the flow through the neck is turbulent).

From the Bernoulli equation the following can be provided,

$$\frac{{p}_{1}}{ρ} + \frac{1}{2} {V}_{1}^{2} = \frac{{p}_{e}}{ρ} + \frac{1}{2} {V}_{c}^{2} + f \frac{{L}_{c}}{{D}_{c}} \frac{{V}_{r}^{2}}{2} ; \qquad ( 15 )$$

where the last term on the right-hand side accounts for viscous losses in the contraction. The kinetic energy can be calculated using the velocity in the wave's reference frame, but the loss term can use the lab frame velocity because the motion of the walls should be taken into account to estimate viscous losses. So, Vᵣ=V_{c}−V₁ can be used. From the conservation of volume, the following expression can be provided,

A₁V₁=A_{c}V_{c}=A₂V₂

The friction factor, f, depends on the Reynolds number (“Re”) of the flow through the neck. For low Re laminar flows, the friction factor can be f=64/Re as an example, and in the inertial flow regime (e.g., Re>>1), the friction factor can be assumed to be constant. The relationship between pressure and area (i.e., tube law) has the form,

$$p = K \frac{Δ A}{{A}_{0}} = K ( \frac{A - {A}_{0}}{{A}_{0}} ) .$$

For a low Reynolds number approximation, flow through the neck can be modeled as,

$${ψ}_{k} = ( \frac{{A}_{1} - {A}_{0}}{{A}_{0}} ) + 1 = {ψ}_{k} ( \frac{{A}_{c} - {A}_{c 0}}{{A}_{c 0}} ) + {( \frac{{A}_{1}}{{A}_{c}} )}^{2} + ψ \frac{{L}_{c}^{′}}{{D}_{c}^{′}} ( \frac{{A}_{1}}{{A}_{c}} - 1 ) ;$$

where A_{c0} represents the reference area of contraction and is much smaller than A₀, and,

$${ψ}_{k} = \frac{K}{\frac{1}{2} ρ {V}_{1}^{2}} ;$$

$${ψ}_{f} = {f}_{W} \frac{L}{{D}_{0}} ;$$

$${f}_{W} = \frac{64 μ}{ρ {V}_{1} {D}_{c}} ;$$

$${L}_{c}^{′} = \frac{{L}_{c}}{L} ; and$$

$${D}_{c}^{′} = \frac{{D}_{c}}{{D}_{0}} .$$

Thus, a reduction in the reference area at some location along the tube length indicates that the tube segment belongs to the contraction zone.

For a high Reynolds number approximation, flow through the neck can be modeled as,

$${ψ}_{k} = ( \frac{{A}_{1} - {A}_{0}}{{A}_{0}} ) + 1 = {ψ}_{k} ( \frac{{A}_{c} - {A}_{c 0}}{{A}_{c 0}} ) + {( \frac{{A}_{1}}{{A}_{c}} )}^{2} + {\underbrace{f \frac{L}{{D}_{0}}}}_{{Ψ}_{f}} {( \frac{{A}_{1}}{{A}_{c}} - 1 )}^{2} \frac{{L}_{c}^{′}}{{D}_{c}^{′}}$$

where f is assumed to be a constant and does not depend on the local Reynolds number in the tube. If it is assumed that there is no viscous loss as the fluid enters the narrower chamber, then the model can be given as,

$${ψ}_{k} = ( \frac{{A}_{1} - {A}_{0}}{{A}_{0}} ) + 1 = {ψ}_{k} ( \frac{{A}_{2} - {A}_{0}}{{A}_{0}} ) + {( \frac{{A}_{1}}{{A}_{2}} )}^{2} + f \frac{{L}_{c}}{{D}_{c}} \frac{{V}_{r}^{2}}{{V}_{1}^{2}} ; \qquad ( 16 )$$

The last term in Eqn. (16) can be selected as,

$$f \frac{{L}_{c}}{{D}_{c}} \frac{{V}_{r}^{2}}{{V}_{1}^{2}} = \{ \begin{matrix}{ψ}_{f} \frac{{L}_{c}^{′}}{{D}_{c}^{′}} ( \frac{{A}_{1}}{{A}_{c}} - 1 ) & for low Re \\ {Ψ}_{f} \frac{{L}_{c}^{′}}{{D}_{c}^{′}} ( \frac{{A}_{1}}{{A}_{c}} - 1 ) & for high Re\end{matrix} .$$

As the peristaltic wave travels over the tube, it naturally leads to a separation of the tube into two parts. One is the length of the tube over which the wave has already traveled and the other is the section of the tube that lies ahead of the contraction. In some implementations, it can be assumed that the tube areas in each of these sections is constant and the ratio of these areas is A₂/A₁ from in FIG. 8. This ratio A₂/A₁ [symbol] x can be solved for as follows.

Letting A_{c}=θA₁, from Eqn. (16) the following equations can be provided for each type of flow,

$${ψ}_{k} \frac{{A}_{1}}{{A}_{0}} {x}^{3} + {x}^{2} ( {\underbrace{{ψ}_{fc} ( \frac{1 - θ}{θ} )}}_{{ψ}_{f}^{′}} - 1 - {ψ}_{k} \frac{{A}_{1}}{{A}_{0}} ) + 1 = 0 for low Re flow ;$$

$$and$$

$${ψ}_{k} \frac{{A}_{1}}{{A}_{0}} {x}^{3} + {x}^{2} ( {\underbrace{{{Ψ}_{fc} ( \frac{1 - θ}{θ} )}^{2}}}_{{Ψ}_{f}^{′}} - 1 - {\underbrace{{ψ}_{k} \frac{{A}_{1}}{{A}_{0}}}}_{{ψ}_{k}^{′}} ) + 1 = 0 for high Re flow .$$

With the introduction of the new variables ψ′_{f} and Ψ′_{f}, the effects of contraction strength and the viscous flow resistance term can be combined. The equation for low Re flow can thus be written as,

ψ′x³+x²(ψ′_{f}−ψ′ₖ−1)+1=0 (17);

and the equation for high Re flow can be written as,

ψ′x³+x²(ψ′_{f}−ψ′ₖ−1)+1=0 (18).

The difference between Eqns. (17) and (18) is the way in which ψ′_{f} and Ψ′_{f} are calculated from the input parameters. For the sake of clarity, the variables references above are summarized again in Table 1.

TABLE 1

| Symbol | Description |
|---|---|
| V₁ | Wave velocity |
| V_{c} | Flow speed in the neck (wave frame) |
| Vᵣ = V_{c} − V₁ | Flow speed in the next (lab frame) |
| f_{W} = (64μ)/(ρV₁D_{c}) | Low Re friction factor |
| ψ_{f} = f_{W} (L/D₀) | Low Re fluid resistance |
| ψₖ = K/(ρV₁²/2) | Inverse of Mach number squared |
| L′_{c} = L_{c}/L | Ratio of neck length to tube length |
| D′_{c} = D_{c}/D₀ | Ratio of neck diameter to the reference diameter |
| θ = A_{c}/A₁ | Area reduction factor for the contraction |
| ψ_{fc} = ψ_{f} (L′_{c}/D′_{c}) | Combined flow resistance term for low Re flow |
| ψ′_{f} = ψ_{fc} (1 − θ)/θ | All-encompassing flow resistance term |
| ψ′ₖ = ψₖ (A₁/A₀) | Tube stiffness and fill volume combination |
| Ψ_{f} = f (L/D₀) | Flow resistance term for high Re flow |
| Ψ′_{f} and ψ′_{f} | Cumulative flow resistance terms |

The two terms ψ′ₖ and ψ′_{f} (or Ψ′_{f}) together account for all of the relevant parameters of the system. The effect of tube stiffness, fluid density, wave velocity, and the amount of fill volume of the tube is accounted for in ψ′ₖ, and the term ψ′_{f} accounts for the fluid's resistance to flow in the tube, the length of the contraction zone, and its intensity as well. All of these effects contribute to the increase or decrease of the area ratio. Typically, to visualize the effect of these variables on pumping patterns, dozens of plots or a plot that has multiple axes (each corresponding to wave velocity, tube stiffness, fluid density, etc.) would be needed. Using the systems and methods described in the present disclosure, these variables can be combined in a sensible manner using the analysis described above, which advantageously enables the visualization of observed regimes in a coherent and clear way.

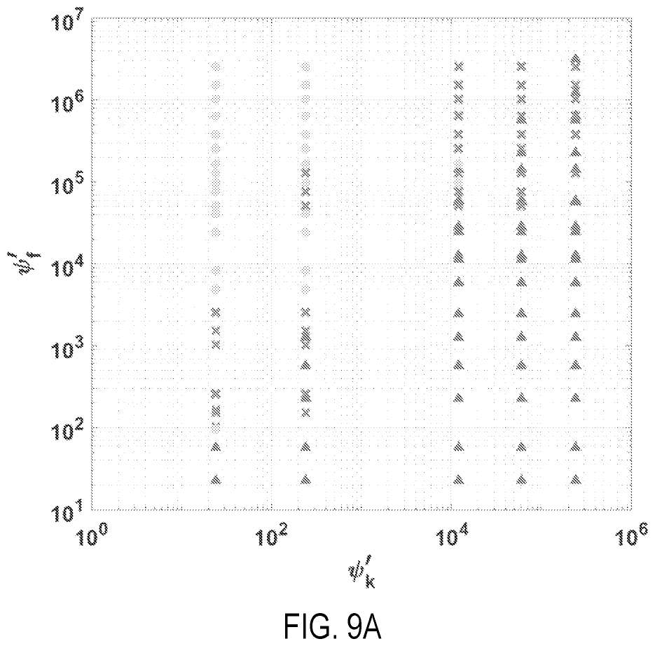

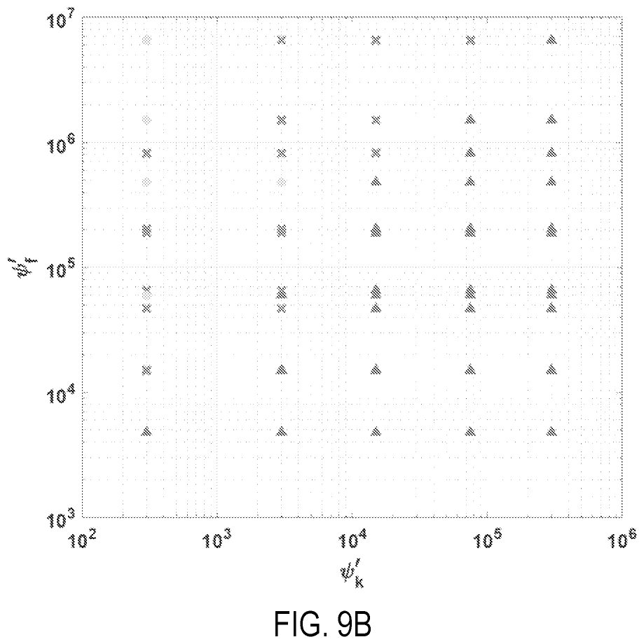

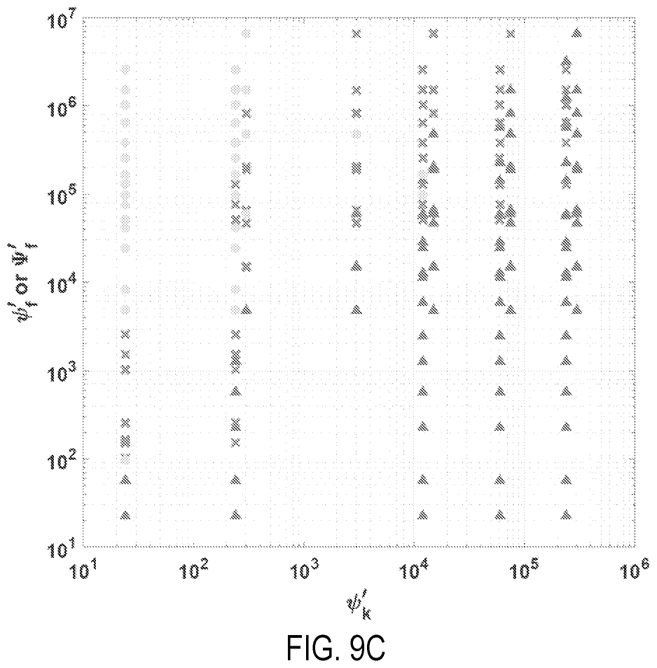

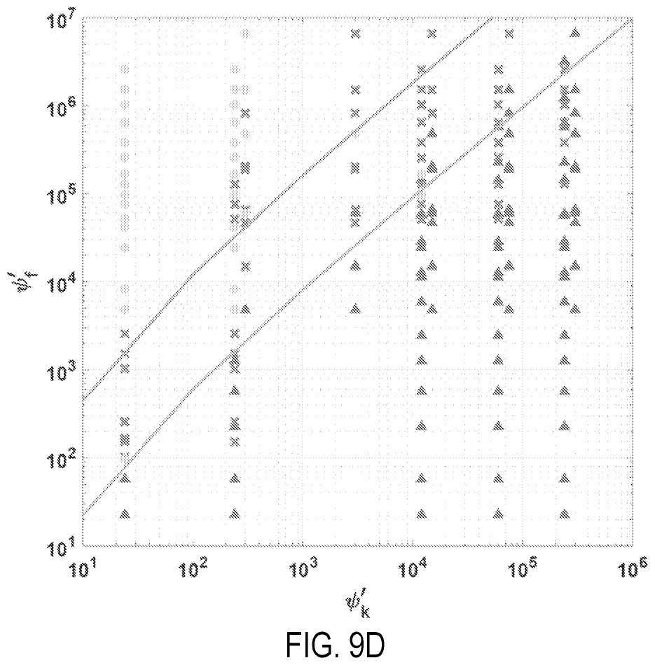

Based on this simplification, the occurrence of each regime can be presented as a set of points on a plot of ψ′_{f} versus ψ′ₖ. FIGS. 9A and 9B show examples of the occurrence of the various regimes at different values of ψ′_{f} and ψ′ₖ, with FIG. 9A showing a regime map for high Re flow and FIG. 9B showing a regime map for low Re flow. FIG. 9C shows an example where the regime points from the two different flow types are combined and presented in a single plot. In this example, it can be seen that the regime data points from both flow types fall within the general vicinity of each other. The set of points belonging to each regime is represented by a specific marker. All combinations of ψ′_{f} and ψ′ₖ for regime 1 are represented by red triangles, regime 2 points are represented by purple crosses, and regimes 3 and 4 are represented by green circles.

From these plots, a general boundary between each set of regime points can be discerned. The boundary represents the change of the system from one solution to another. As such, it is contemplated that the discriminant of the cubic equations given by Eqns. (17) and (18) can predict the slope of this boundary. Regime 1 corresponds to x≈1 and Regime 2 corresponds to x≈0. When the discriminant of this equation is zero, the following relationship between ψ′_{f} and ψ′ₖ is provided,

$${ψ}_{f}^{′} = {ψ}_{k}^{′} - 3 {( \frac{{ψ}_{k}^{′}}{2} )}^{2 / 3} + 1. \qquad ( 19 )$$

Plotting this equation on the regime map given in FIG. 9C results in the lower slope in FIG. 9D. Adding an offset to this slope (e.g., by a factor of 10) can be used to match the exact boundary as seen the regime map with the curve plotted using Eqn. (19). The boundary between regimes 2 and 3 can also be demarcated by another offset (e.g., using a factor of 10). The combined regime map with these boundaries is shown in FIG. 9D.

Using these regime maps, the shape assumed by the tube during peristalsis can be predicted or otherwise estimated by computing the relevant ψ′_{f} and ψ′ₖ from the given operating conditions and finding the region to which these points belong.

The regime maps also help understand the change in tube shape that would be observed as one of the operating conditions is changed. For instance, as the tube stiffness increases, the system will deform in such a way that the area ratio, x, tends to 1.0. Increasing the fill volume or decreasing the peristaltic wave velocity leads to a similar outcome. When it comes to increasing the fluid's viscosity, a transition from Regime 1 to Regime 2 can be observed, as predicted by the regime map when the value of ψ′_{f} is increased. When the area contraction factor, θ, is reduced (i.e., the amount of wave “squeezing” is increased), the system transition from Regime 1 to Regime 2 can also be observed.

Each of these regimes corresponds to a specific deformation pattern observed in the dilation catheter device in different patients and/or diseases. For instance, in a non-limiting example, it can be assumed that the device is calibrated and the operating conditions are benchmarked in such a way that it shows pumping patterns corresponding to Regime 2 in a healthy individual. Under the same operating conditions, a patient with a diseased esophagus will display pumping patterns corresponding to either Regime 1 or Regime 3/4. Depending on the specific behavior displayed, the regime map helps identify the exact cause of the abnormality. For instance, a patient displaying regime 1 might have a stiffer esophagus due to fibrosis and a patient with Regime 4 can be said to be suffering from dysphagia due to ineffective peristalsis. Thus, the quantification of the device's behavior in the form of these regime maps can be used to directly assist in better interpreting the various shapes seen in patients during the a procedure using a dilation catheter.

When the value of 0 approaches 1, the value of ψ′_{f} goes to zero. As such, the regimes maps shown in FIGS. 9A-9D do not display this region of operation. The complete regime map would show that the regions occupied by regimes 1 and 2 would be flanked by points from Regime 4 not only at the top (as is shown in FIGS. 9A-9D), but on the bottom as well. The inclusion of these points for the lower values of ψ′_{f} would unnecessarily clutter the regime plot and were thus excluded for clarity.

Advantageously, the systems and methods described in the present disclosure can provide an analysis that differentiates “effective” and “ineffective” peristaltic contraction waves. For instance, determining the work done by a peristaltic wave and observing its variation during pumping can offer further insights on the pumping process.

In the examples described below, the configuration that is being analyzed is a closed system. As such, there is no “net flow rate” or non-zero displacement of fluid volume over one peristaltic event, which can be used to quantify efficacy. At the end of peristalsis, all the work done by the peristaltic wave is dissipated due to fluid viscosity. So, the amount of energy dissipated is the spent pumping work. During peristalsis however, the stretching of the tube walls leads to some of the spent energy being stored as elastic potential energy. After the passage of the peristaltic wave, the tube relaxes and releases all the energy back into the fluid, which in turn is lost via viscous dissipation. To understand the quantitative relationship between these three agents, the following model can be used:

$${\underbrace{\frac{\partial }{\partial t} {{\int }^{L}}_{0} ( \frac{1}{2} ρ {Au}^{2} ) dx}}_{Rate of change in K . E .} + {\underbrace{{{\int }^{L}}_{0} p \frac{\partial A}{\partial t} dx}}_{Work done by fluid on the tube walls} + {\underbrace{{{\int }^{L}}_{0} 8 πμ {u}^{2} dx}}_{Viscous dissipation} = 0 ; \qquad ( 20 )$$

where a zero velocity condition Q=u=0 is applied at the tube ends. This equation succinctly shows how the power is distributed in the system at each time instant. When the pressure term is put on the right-hand side, it gets a negative sign and then represents the work done by the walls on the fluid. The left-hand side then shows that part of that energy goes into changing the kinetic energy of the fluid and the rest is lost via viscous dissipation. It is important to realize that the pressure term has contributions from both the passive elastic part of the tube and the rise due to active contraction. When separated, the following understanding of the power breakdown is achieved:

$$\frac{\partial }{\partial t} {\int }_{0}^{L} ( \frac{1}{2} ρ A {u}^{2} ) dx + {\int }_{0}^{L} {p}_{p a s s} \frac{\partial A}{\partial t} dx + {\int }_{0}^{L} 8 π μ {u}^{2} dx = - {\int }_{0}^{L} {p}_{actv} \frac{\partial A}{\partial t} dx . \qquad ( 21 )$$

In this form, the right-hand side is the rate of work done by the active part of the tube wall (the peristaltic contraction) on the confined fluid and the terms on the left-hand side show the consumers of this spent power. Some of it goes into increasing the kinetic energy of the fluid, some of it is stored in the tube walls when they stretch due to an increase in local pressure, and the rest is lost via dissipation. At this point, some form of the passive pressure can be assumed in order to compute the values of each these terms from the various regimes displayed in the 1D model. One non-limiting example of the passive pressure component is a linear dependence on the tube area. The breakdown of the fluid pressure can then be presented as,

$$P = \frac{α}{θ} - 1 = {\underbrace{( α - 1 )}}_{passive} + {\underbrace{α ( \frac{1}{θ} - 1 )}}_{active} . \qquad ( 22 )$$

Using this or another suitable breakdown of the fluid pressure term, the values of each of the terms in Eqn. (21) can be computed and the variation of work done or energy dissipated over a single peristaltic event for each of the reported regimes can be visualized. The above analysis can also be repeated for the momentum equation with the friction factor stress term.

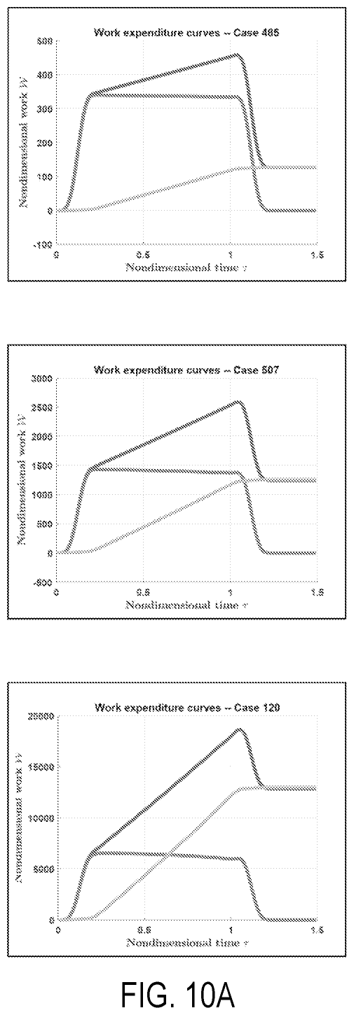

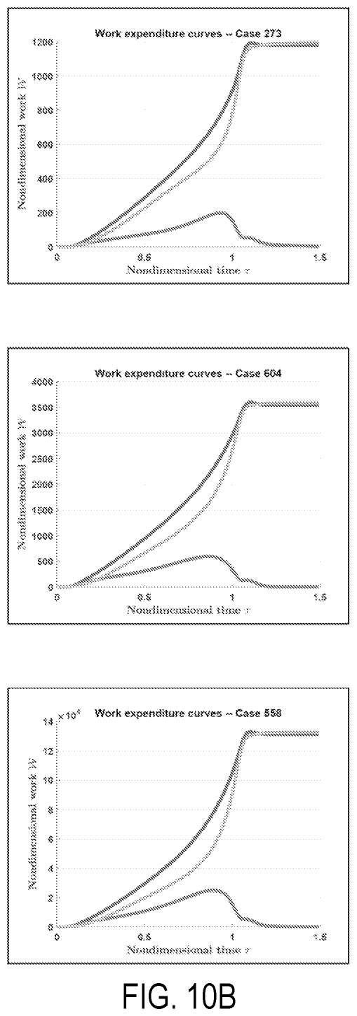

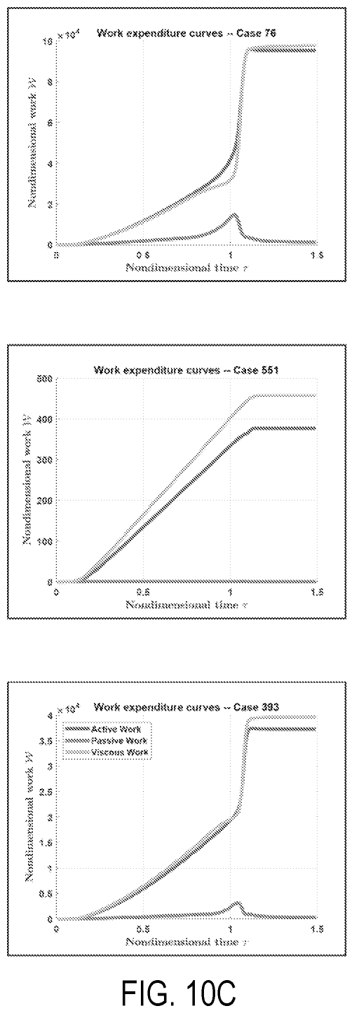

As described above, Eqn. (21) gives the balance of power at every instant of time. Integrating this equation over time gives the balance of work done or energy lost as the peristaltic wave advances. FIGS. 10A-10C show the energy contribution from each of these terms across all the observed regimes (Regime 1: FIG. 10A, Regime 2: FIG. 10B, Regimes 3, 4: FIG. 10C) as computed using the reduced order model described in the present disclosure. These figures summarize the energy transfer pathways between the various sinks over the entire range of observed pumping patterns. At time τ=0, the peristaltic wave begins traveling over the tube length and the active work done by it is zero. As the wave advances, the work done by it (i.e., the active work) is split into either increasing the potential energy stored in the tube walls or generating flow fields that then lose energy via dissipation.

The work curves for each of these regimes show unique identifying features that confirm what is visually observed through the tube deformation patterns. For Regime 1, once the wave has created a zone of reduced area, the active work goes into overcoming the viscous resistance and the potential energy of the walls remains unchanged. Upon approaching the right boundary, the tube relaxes and the stored elastic energy is recovered. For Regime 2, a gradual rise in stored elastic energy with time is observed, indicating that the activation wave is continuously doing work on the tube wall. Unlike Regime 1, when the wave approaches the end and allows the tube walls to relax, the stored energy is lost via fluid dissipation. This is observed by following the viscous work curve, which sharply rises around the τ=0.8 mark to meet the active work curve. In spite of large tube wall deformations associated with this regime, the majority of active work done is still lost via viscous dissipation. Regimes 3 and 4 reflect the low wall deformations observed from the tube shape plots. The passive work done is extremely small compared to the active work done, which is almost entirely lost. It should be noted that the magnitude of active work should generally be the largest among the three curves, but in light of numerical errors the viscous work may appear to be larger. This can be understood by non-dimensionalizing Eqn. (21),

$$\frac{\partial }{\partial t} {\int }_{0}^{1} ( \frac{1}{2} α {U}^{2} ) d χ + ψ {\int }_{0}^{1} {P}_{p a s s} \frac{\partial α}{\partial τ} d χ + β {\int }_{0}^{1} {U}^{2} d χ = - ψ {\int }_{0}^{1} {P}_{actv} \frac{\partial α}{\partial τ} d χ . \qquad ( 23 )$$

For Regimes 3 and 4, the value of β is quite large compared to ψ, so the computation of viscous work involves the product of a large and small number, which can lead to the discrepancy mentioned above. An extremely high value of β impedes any change in A, which can directly lead to almost no fluid flow or tube deformation.

This detailed look into the variation of the active, passive, and viscous work done during a peristaltic event provides analytical tools to identify healthy pumping waves and the conditions under which reasonable tube wall deformations may be observed. A significant change in the tube area for this configuration due to peristalsis indicates that the wave has some ability to move fluid forward in a normal setting, which involves bolus transport following healthy deglutition.

The reduced order model described in the present disclosure takes as input the activation wave and predicts the resultant tube wall deformation and the associated fluid velocity and pressure fields. Data acquired with a balloon dilation catheter contains the variation of tube wall deformation as a function of time and the value of pressure at a single location, such as the (approximate) location x=χL=16 cm in some example configurations. The values of fluid velocity and pressure at all the other locations along the tube are unknown. To tackle this issue, the continuity equation is used to obtain the gradient of velocity field and then the momentum equation is used and differentiated with respect to χ in order to obtain a second order derivative of velocity, U. This allows for the application of the zero-velocity boundary conditions at both ends. The pressure reading obtained from the patient data can be applied as a pinning condition at the χ=1 boundary. Following that, this equation can be used to determine the velocity and pressure fields as a function of χ and τ. In the following paragraphs, the mathematical details of this process are described.

First, the same non-dimensional numbers as defined above are used, except for pressure, which is non-dimensionalized using ρc². In this way, the following similar set of non-dimensional governing equations can be determined, but with a single non-dimensional number, β=(8πμL)/ρA₀c):

$$\frac{\partial α}{\partial τ} + \frac{\partial q}{\partial χ} = 0 ; \qquad ( 24 )$$

$$\frac{\partial q}{\partial τ} + \frac{\partial }{\partial χ} ( \frac{{q}^{2}}{α} ) + α \frac{\partial p}{\partial χ} + β \frac{q}{α} = 0. \qquad ( 25 )$$

In this form, this system of equations does not allow for specification of boundary conditions at both ends of the domain. To address this, the spatial derivative of Eqn. (24) can be computed to result in,

$$\frac{{\partial }^{2} α}{\partial τ \partial χ} + \frac{{\partial }^{2} q}{\partial {χ}^{2}} = 0 . \qquad ( 26 )$$

The non-dimensional area, α, is obtained from the balloon dilatation catheter data and q can be solved for implicitly (Poisson equation) assuming the boundary condition q=0 at χ=0, 1. The momentum equation can then be used to calculate p using the given values of α and the newly computed q values. The boundary condition for p at χ=1 can be obtained from a distal pressure sensor on the balloon dilation catheter. A zero pressure gradient condition can also be applied at the χ=0 boundary. Just like the continuity equation, the momentum equation can be differentiated with respect to χ and this equation used to compute p implicitly,

$$\frac{{\partial }^{2} q}{\partial χ \partial τ} + \frac{{\partial }^{2}}{{\partial }^{2} χ} ( \frac{{q}^{2}}{α} ) + \frac{\partial }{\partial χ} ( α \frac{\partial p}{\partial χ} ) + β \frac{\partial }{\partial χ} ( \frac{q}{α} ) = 0 . \qquad ( 27 )$$

As noted above, in some instances it may be desirable to model the presence of the catheter. The diameter readings reported by the planimetry sensors do not correspond to the area through which fluid flows. The fluid flows between the reported diameter values and the diameter of the catheter. Several possible approaches can be taken to account for the presence of the catheter. As one example, the catheter's diameter can be subtracted from the reported diameter values. As another example, the flow can be considered to be annular and the mathematical formulation of the model can be updated by changing the viscous flow stress term to account for the inner (i.e., catheter) diameter.

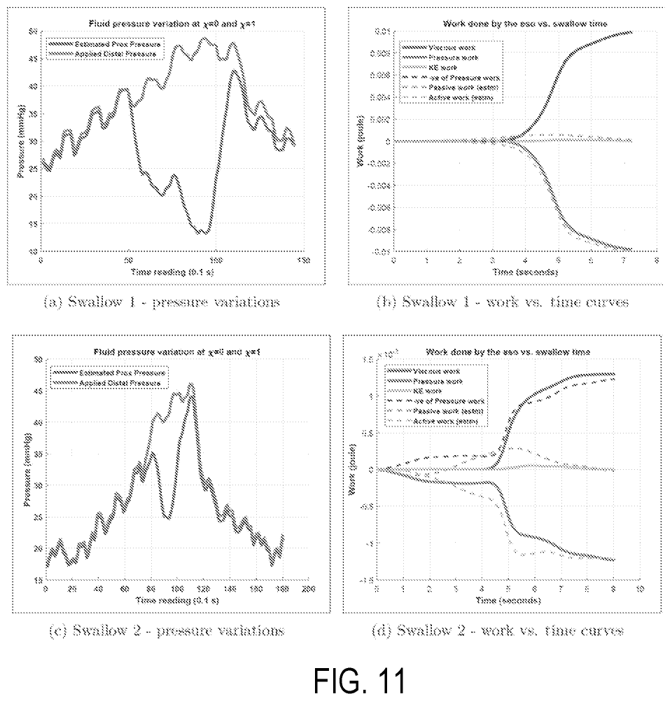

In an example study, the systems and methods described in the present disclosure were implemented to generate work curves from data acquired using a balloon dilation catheter. A particular contraction wave to study was isolated from acquired data and work curves were generated for that contraction wave. A window of readings that covered a single pressure peak was selected, such as readings from 50 to 150. At the first reading (#50), the wave began to travel over the tube length and resulted in a pressure rise. The wave was traveling as time went on and by the last reading (#150) the wave had finished traveling over the entire tube length. In FIG. 11, the work curves and the pressures at χ=0 (referred to as the proximal pressure) and χ=1 (referred to as the distal pressure) as a function of time for two typical swallows are shown. The former is predicted by the reduced order model and the latter is applied to the model to fix the pressure levels inside the tube.

One thing to note from the analysis of the patient swallow data is the pressure predicted by the model at the proximal (left) end of the tube at χ=0. When the wave is incoming, the pressure in the entire system rises, but once it passes over the left end, there is a sharp drop in pressure. This is due to the temporary displacement of fluid due to the peristaltic pumping action of the wave leading to the tube to be locally deflated at the left end. The displaced fluid then stretches the walls of the distal (right) end of the tube at χ=1, and this is marked by the continuous rise in pressure at that location. Once the wave has passed, the fluid accumulated at the end rushes back and the tube attains a uniform shape and the pressures at both locations become equal at the end of wave travel.

The work curves shown in the right column of FIG. 11 were computed from the estimated fluid pressure and velocity fields. The curves show the same behavior observed in the curves derived from the 1D model (i.e., the work done to change the kinetic energy of the fluid is minimal and the active work done is mostly lost via viscous dissipation). This is seen by the near overlap of the viscous curve and the curve showing the negative of active work done. The sign of each of the work curves indicates if the term is a source or a sink. The energy sinks are given a positive sign and the sources have a negative sign. Their sum should equal to zero (approximately) and that is what is observed in the work curves for both swallows.

The calculation of active and passive work done from the 1D model depends on the value of θ, which is completely known. However, the activation strength may not be available from real-world patient data, as such estimating the breakdown of the pressure work term into an active and passive component can in some instances difficult. To address this, we use the balloon dilation catheter data are used to determine the tube's reference area and the tube stiffness constant when the esophagus is fully relaxed. Once this information is obtained, the passive power can be estimated as follows,

$${P}_{p a s s} = {\int }_{0}^{L} {p}_{p a s s} \frac{\partial A}{\partial t} d x = {\int }_{0}^{L} K ( \frac{A}{{A}_{0}} - 1 ) \frac{\partial A}{\partial t} dx \qquad ( 28 )$$

The active work was then estimated by subtracting the passive work from the total fluid pressure work. These estimated values of passive and active work are plotted in the work curves using dotted lines.

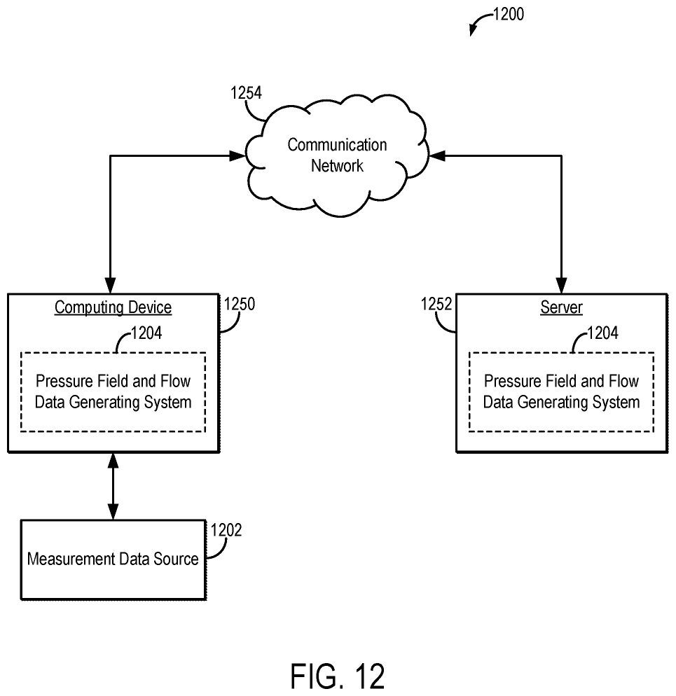

Referring now to FIG. 12, an example of a system 1200 for generating pressure field data, fluid velocity or other flow data, and/or muscular work data from planimetry and pressure data acquired with a catheter, such as a balloon dilation or other planimetry catheter, in accordance with some embodiments of the systems and methods described in the present disclosure is shown. As shown in FIG. 12, a computing device 1250 can receive one or more types of data (e.g., planimetry data, pressure data) from measurement data source 1202, which may be a planimetry and pressure measurement data source. In some embodiments, computing device 1250 can execute at least a portion of a pressure field and flow data generating system 1204 to generate pressure field data, fluid velocity or other flow data, and/or muscular work data from planimetry and pressure data received from the measurement data source 1202.

Additionally or alternatively, in some embodiments, the computing device 1250 can communicate information about data received from the measurement data source 1202 to a server 1252 over a communication network 1254, which can execute at least a portion of the pressure field and flow data generating system 1204. In such embodiments, the server 1252 can return information to the computing device 1250 (and/or any other suitable computing device) indicative of an output of the pressure field and flow data generating system 1204.

In some embodiments, computing device 1250 and/or server 1252 can be any suitable computing device or combination of devices, such as a desktop computer, a laptop computer, a smartphone, a tablet computer, a wearable computer, a server computer, a virtual machine being executed by a physical computing device, and so on. The computing device 1250 and/or server 1252 can also reconstruct images from the data.

In some embodiments, measurement data source 1202 can be any suitable source of image data (e.g., measurement data, images reconstructed from measurement data), such as a planimetry catheter, another computing device (e.g., a server storing image data), and so on. In some embodiments, measurement data source 1202 can be local to computing device 1250. For example, measurement data source 1202 can be incorporated with computing device 1250 (e.g., computing device 1250 can be configured as part of a device for capturing, scanning, and/or storing images). As another example, measurement data source 1202 can be connected to computing device 1250 by a cable, a direct wireless link, and so on. Additionally or alternatively, in some embodiments, measurement data source 1202 can be located locally and/or remotely from computing device 1250, and can communicate data to computing device 1250 (and/or server 1252) via a communication network (e.g., communication network 1254).

In some embodiments, communication network 1254 can be any suitable communication network or combination of communication networks. For example, communication network 1254 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, and so on. In some embodiments, communication network 1254 can be a local area network, a wide area network, a public network (e.g., the Internet), a private or semi-private network (e.g., a corporate or university intranet), any other suitable type of network, or any suitable combination of networks. Communications links shown in FIG. 12 can each be any suitable communications link or combination of communications links, such as wired links, fiber optic links, Wi-Fi links, Bluetooth links, cellular links, and so on.

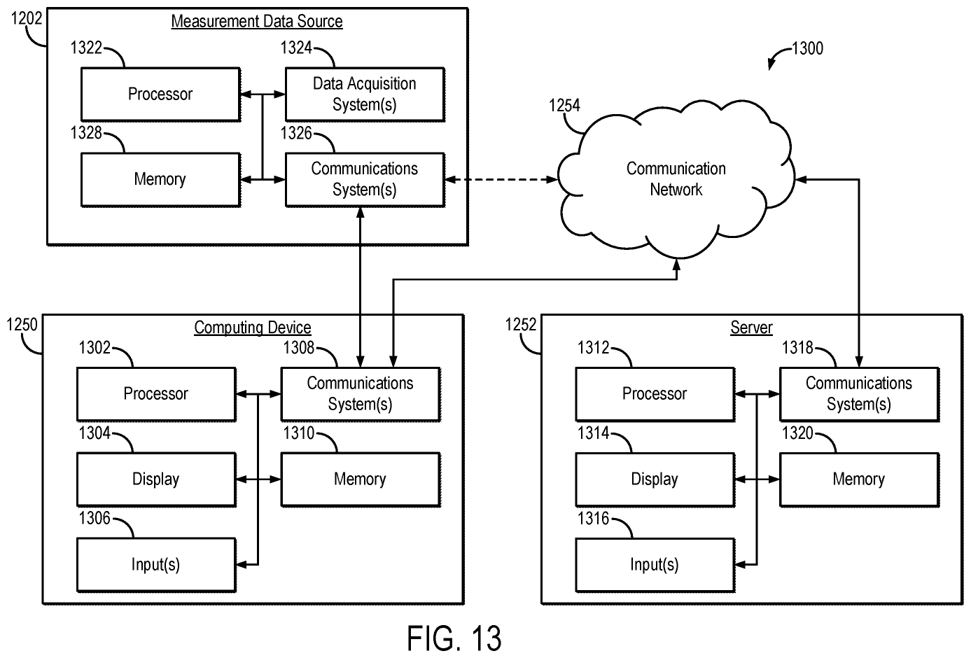

Referring now to FIG. 13, an example of hardware 1300 that can be used to implement measurement data source 1202, computing device 1250, and server 1252 in accordance with some embodiments of the systems and methods described in the present disclosure is shown. As shown in FIG. 13, in some embodiments, computing device 1250 can include a processor 1302, a display 1304, one or more inputs 1306, one or more communication systems 1308, and/or memory 1310. In some embodiments, processor 1302 can be any suitable hardware processor or combination of processors, such as a central processing unit (“CPU”), a graphics processing unit (“GPU”), and so on. In some embodiments, display 1304 can include any suitable display devices, such as a computer monitor, a touchscreen, a television, and so on. In some embodiments, inputs 1306 can include any suitable input devices and/or sensors that can be used to receive user input, such as a keyboard, a mouse, a touchscreen, a microphone, and so on.

In some embodiments, communications systems 1308 can include any suitable hardware, firmware, and/or software for communicating information over communication network 1254 and/or any other suitable communication networks. For example, communications systems 1308 can include one or more transceivers, one or more communication chips and/or chip sets, and so on. In a more particular example, communications systems 1308 can include hardware, firmware and/or software that can be used to establish a Wi-Fi connection, a Bluetooth connection, a cellular connection, an Ethernet connection, and so on.

In some embodiments, memory 1310 can include any suitable storage device or devices that can be used to store instructions, values, data, or the like, that can be used, for example, by processor 1302 to present content using display 1304, to communicate with server 1252 via communications system(s) 1308, and so on. Memory 1310 can include any suitable volatile memory, non-volatile memory, storage, or any suitable combination thereof. For example, memory 1310 can include RAM, ROM, EEPROM, one or more flash drives, one or more hard disks, one or more solid state drives, one or more optical drives, and so on. In some embodiments, memory 1310 can have encoded thereon, or otherwise stored therein, a computer program for controlling operation of computing device 1250. In such embodiments, processor 1302 can execute at least a portion of the computer program to present content (e.g., images, user interfaces, graphics, tables), receive content from server 1252, transmit information to server 1252, and so on.

In some embodiments, server 1252 can include a processor 1312, a display 1314, one or more inputs 1316, one or more communications systems 1318, and/or memory 1320. In some embodiments, processor 1312 can be any suitable hardware processor or combination of processors, such as a CPU, a GPU, and so on. In some embodiments, display 1314 can include any suitable display devices, such as a computer monitor, a touchscreen, a television, and so on. In some embodiments, inputs 1316 can include any suitable input devices and/or sensors that can be used to receive user input, such as a keyboard, a mouse, a touchscreen, a microphone, and so on.

In some embodiments, communications systems 1318 can include any suitable hardware, firmware, and/or software for communicating information over communication network 1254 and/or any other suitable communication networks. For example, communications systems 1318 can include one or more transceivers, one or more communication chips and/or chip sets, and so on. In a more particular example, communications systems 1318 can include hardware, firmware and/or software that can be used to establish a Wi-Fi connection, a Bluetooth connection, a cellular connection, an Ethernet connection, and so on.

In some embodiments, memory 1320 can include any suitable storage device or devices that can be used to store instructions, values, data, or the like, that can be used, for example, by processor 1312 to present content using display 1314, to communicate with one or more computing devices 1250, and so on. Memory 1320 can include any suitable volatile memory, non-volatile memory, storage, or any suitable combination thereof. For example, memory 1320 can include RAM, ROM, EEPROM, one or more flash drives, one or more hard disks, one or more solid state drives, one or more optical drives, and so on. In some embodiments, memory 1320 can have encoded thereon a server program for controlling operation of server 1252. In such embodiments, processor 1312 can execute at least a portion of the server program to transmit information and/or content (e.g., data, images, a user interface) to one or more computing devices 1250, receive information and/or content from one or more computing devices 1250, receive instructions from one or more devices (e.g., a personal computer, a laptop computer, a tablet computer, a smartphone), and so on.

In some embodiments, measurement data source 1202 can include a processor 1322, one or more data acquisition systems 1324, one or more communications systems 1326, and/or memory 1328. In some embodiments, processor 1322 can be any suitable hardware processor or combination of processors, such as a CPU, a GPU, and so on. In some embodiments, the one or more data acquisition systems 1324 are generally configured to acquire data, images, or both, and can include a planimetry catheter. Additionally or alternatively, in some embodiments, one or more data acquisition systems 1324 can include any suitable hardware, firmware, and/or software for coupling to and/or controlling operations of a planimetry catheter. In some embodiments, one or more portions of the one or more data acquisition systems 1324 can be removable and/or replaceable.

Note that, although not shown, measurement data source 1202 can include any suitable inputs and/or outputs. For example, measurement data source 1202 can include input devices and/or sensors that can be used to receive user input, such as a keyboard, a mouse, a touchscreen, a microphone, a trackpad, a trackball, and so on. As another example, measurement data source 1202 can include any suitable display devices, such as a computer monitor, a touchscreen, a television, etc., one or more speakers, and so on.

In some embodiments, communications systems 1326 can include any suitable hardware, firmware, and/or software for communicating information to computing device 1250 (and, in some embodiments, over communication network 1254 and/or any other suitable communication networks). For example, communications systems 1326 can include one or more transceivers, one or more communication chips and/or chip sets, and so on. In a more particular example, communications systems 1326 can include hardware, firmware and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 1328 can include any suitable storage device or devices that can be used to store instructions, values, data, or the like, that can be used, for example, by processor 1322 to control the one or more data acquisition systems 1324, and/or receive data from the one or more data acquisition systems 1324; to images from data; present content (e.g., images, a user interface) using a display; communicate with one or more computing devices 1250; and so on. Memory 1328 can include any suitable volatile memory, non-volatile memory, storage, or any suitable combination thereof. For example, memory 1328 can include RAM, ROM, EEPROM, one or more flash drives, one or more hard disks, one or more solid state drives, one or more optical drives, and so on. In some embodiments, memory 1328 can have encoded thereon, or otherwise stored therein, a program for controlling operation of measurement data source 1202. In such embodiments, processor 1322 can execute at least a portion of the program to generate images, transmit information and/or content (e.g., data, images) to one or more computing devices 1250, receive information and/or content from one or more computing devices 1250, receive instructions from one or more devices (e.g., a personal computer, a laptop computer, a tablet computer, a smartphone, etc.), and so on.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

The present disclosure has described one or more preferred embodiments, and it should be appreciated that many equivalents, alternatives, variations, and modifications, aside from those expressly stated, are possible and within the scope of the invention.

1. A method for generating flow data from planimetry data acquired with a planimetry catheter positioned within a lumen, the method comprising:
(a) accessing planimetry data with a computer system, the planimetry data being acquired with a planimetry catheter;
(b) accessing pressure data with the computer system, the pressure data being acquired with the planimetry catheter;
(c) inputting the planimetry data and the pressure data to a reduced order model using the computer system, generating output as flow data; and
(d) storing the flow data in a memory of the computer system.

2. The method of claim 1, wherein the reduced order model comprises a one-dimensional fluid mechanics-based model.

3. The method of claim 2, wherein the reduced order model incorporates effects of a nonlinear relationship between pressure and area on a value of work done during peristalsis.

4. The method of claim 2, wherein the reduced order model is constructed to model peristaltic activation waves to have a non-constant velocity.

5. The method of claim 1, wherein the flow data comprise fluid velocity data indicative of fluid velocity values along an extent of the planimetry catheter.

6. The method of claim 1, wherein the flow data comprise pressure field data indicative of pressure values along an extent of the planimetry catheter.

7. The method of claim 1, wherein the flow data comprise both pressure field data indicative of pressure values along an extent of the planimetry catheter and fluid velocity data indicative of fluid velocity values along an extent of the planimetry catheter.

8. The method of claim 7, wherein the planimetry data and pressure data are acquired while the planimetry catheter has been positioned within a tubular organ, and further comprising generating with the computer system, muscular work data from the pressure field data and the fluid velocity data, wherein the muscular work data quantify work done by a muscle associated with the tubular organ.

9. The method of claim 1, further comprising classifying the flow data as belonging to one of a plurality of different regimes that each indicate different patterns of peristaltic pumping that are defined based on a response of a wall of the lumen and fluid within the lumen to an applied activation.

10. The method of claim 1, wherein the planimetry data comprise measurements of geometry along an extent of a balloon of the planimetry catheter.

11. The method of claim 10, wherein the planimetry data comprise measurements of area along an extent of a balloon of the planimetry catheter.

12. The method of claim 10, wherein the planimetry data comprise measurements of at least one of internal diameter, cross-section, or distensibility of the lumen into which the planimetry catheter has been positioned.

13. The method of claim 1, wherein the pressure data are measured at a point along an extent of the planimetry catheter.

14. The method of claim 13, wherein the point is adjacent a tip of the planimetry catheter.

15. The method of claim 1, wherein the pressure data are measured at multiple points along an extent of the planimetry catheter.

16. The method of claim 1, further comprising increasing a sampling density of at least one of the planimetry data or the pressure data by interpolating the at least one of the planimetry data or the pressure data to the sampling density.

17. The method of claim 1, further comprising performing volume correction on at least one of the planimetry data or the pressure data using a bag volume of the planimetry catheter as a basis for the volume correction.
